(19) United States
(12) Patent Application Publication
(10) Pub. No.: US 2018/0290138 A1
(43) Pub. Date: Oct. 11, 2018

(54) MICROFLUIDIC CHIP FOR SCREENING ANTICANCER DRUG RESISTANT CELLS AND USE THEREOF

(71) Applicants: RESEARCH & BUSINESS FOUNDATION SUNGKYUNKWAN UNIVERSITY, Suwon-si, Gyeonggi-do (KR); EWHA UNIVERSITY-INDUSTRY COLLABORATION FOUNDATION, Seoul (KR)

(72) Inventors: Sungsu PARK, Seoul (KR); Sanghyuk LEE, Seoul (KR)

(73) Assignees: RESEARCH & BUSINESS FOUNDATION SUNGKYUNKWAN UNIVERSITY, Suwon-si, Gyeonggi-do (KR); EWHA UNIVERSITY-INDUSTRY COLLABORATION FOUNDATION, Seoul (KR)

(21) Appl. No.: 15/573,924

(22) PCT Filed: May 13, 2016
(86) PCT No.: PCT/KR2016/005108

§ 371 Date: Nov. 14, 2017

(30) Foreign Application Priority Data
May 15, 2015 (KR) 10-2015-0068217

(51) Int. Cl. B01L 3/00; G01N 33/50

(52) U.S. Cl. CPC B01L 3/502715; G01N 33/5011; B01L 2200/0694; B01L 2300/0816; B01L 2300/0861; G01N 33/5044

(57) ABSTRACT

The present invention relates to a microfluidic chip for screening anticancer drug resistant cells and a method for inducing or screening anticancer drug resistance using the same. The microfluidic chip of the present invention can induce a continuous concentration gradient between cell culture chambers and can implement the prompt induction and read-out within a week, unlike in existing read-out techniques, and thus, is expected to be able to take a target treatment through more fundament approach, in the treatment of cancer.

### TECHNICAL FIELD

The present invention relates to a microfluidic chip for screening anticancer drug resistant cells and a use thereof, and more particularly, to a microfluidic chip for screening anticancer drug resistant cells, which can create a continuous concentration gradient of an anticancer drug, and a method for inducing or screening anticancer drug resistant cells.

### BACKGROUND ART

Cancer is one of the major causes of death, occupying first or second place, for adults in Korea, and therefore, despite a variety of studies, approximately more than half of cancer patients end up dying. Cancer treatment methods that have been known to be efficient until now are surgery, radiation therapy, and chemotherapy, and there are some differences between them. For example, the surgery and radiation therapy are local treatments effective only on an excised part or an irradiated part, and the chemotherapy is a systemic treatment effective on the entire body.

Generally, cancer is a disease that is generated from a local lesion and spreads to the entire body, and has systemic metastases to a certain degree, except when found in an early stage. For this reason, no matter how effective local treatment is used, there is a high risk of recurrence, suggesting that, for cancer treatment, most cancer patients need chemotherapy, which is a systemic treatment, as well as surgery and radiation therapy, which are local therapies. Therefore, at present, to improve therapeutic effects on cancer, the expansion of radical surgery determined by early diagnosis is needed, and chemotherapy is essential for patients with inoperable cancer or with a high risk of recurrence after surgery.

However, despite the development of a variety of anticancer drugs until now, only a few types of cancer such as leukemia, malignant lymphoma, and testicular cancer are completely curable just with anticancer drugs, this is because cancer cells do not respond to the anticancer drugs in cancer treatment with anticancer drugs, or while the anticancer drugs are effective in shrinking tumors in the initial treatment, cancer patients become resistant to the anticancer drugs during or after the treatment. The resistance to an anticancer drug means that, when cancer patients are treated with an anticancer drug, the drug has no therapeutic effect from the beginning, or is effective in the initial cancer treatment but ends up losing its effect during a continuous treatment process. In this case, since cancer cells may have multi-drug resistance (MDR), which is resistance shown to different types of anticancer drugs as well as an anticancer drug used in treatment, once the cancer cells acquire the resistance to anticancer drugs, there is difficulty in cancer treatment afterwards.

Therefore, for effective chemotherapy, the resistance to anticancer drugs shown by cancer cells should be overcome, and to this end, it is necessary to identify genes related to the resistance to anticancer drugs shown by the cancer cells. Among several anticancer drug resistant genes that have been known so far, the most well-known ones are P-glycoprotein (PgP), a multidrug resistance-associated protein (MRP), etc. Such resistance-associated genes of cancer cells may be used in a novel treatment method in combination with an anticancer drug, and may serve as markers that predict the development of anticancer drug resistance.

Culturing and screening of cancer cells are a prerequisite for analysis of such resistance-associated genes, and thus a variety of studies have been conducted (Korean Unexamined Patent Application Publication No. 10-2013-0156493), but are still not inadequate. Accordingly, the inventors designed a microfluidic chip for screening anticancer drug resistant cells, in which a continuous concentration gradient between cell culture chambers is created and a method for inducing or screening anticancer drug resistant cells using the same to reduce a period of culturing anticancer drug resistant cells, conventionally taking at least 6 months.

### DISCLOSURE

### Technical Problem

In order to solve the above problems, the present inventors confirmed an effect of enhancing anticancer drug resistance of cells cultured in a microfluidic chip of the present invention, and based on this, the present invention was completed.

Therefore, the present invention is directed to providing a microfluidic chip for screening or inducing anticancer drug resistant cells, which comprises: a plate 100 including a plurality of cell culture chambers 110 in a radial shape; a cell introduction part 200 formed in a central area of the plate 100 to load cells; a fluid diffusion part 300 formed along the periphery of the plate 100 to impart a space for the flow of a fluid; a first inlet 400 connected with the fluid diffusion part 300 to inject a fluid containing a culture medium and an anticancer drug; a second inlet 500 connected with the fluid diffusion part 300 to inject a fluid containing a culture medium; micro-channels 600 providing paths for the flow of fluids between the fluid diffusion part 300, the cell culture chambers 110 and the cell introduction part 200; and outlets 700 connected with the fluid diffusion part 300 to discharge the fluids outside.

The present invention is also directed to providing a method for inducing anticancer drug resistant cells using the microfluidic chip, which comprises: loading cancer cells isolated from patients into a cell introduction part 200; injecting a fluid containing a culture medium and an anticancer drug and a fluid containing a culture medium into a first inlet 400 and a second inlet 500, respectively; and forming a concentration gradient between cell culture chambers 110 by passing the fluids through micro-channels 600.

The present invention is also directed to providing a method for screening anticancer drug resistant cells using the microfluidic chip, which comprises loading cancer cells isolated from patients into a cell introduction part 200; injecting a fluid containing a culture medium and an anticancer drug and a fluid containing a culture medium into a first inlet 400 and a second inlet 500, respectively; forming a concentration gradient between cell culture chambers 110 by passing the fluids through micro-channels 600; and real-time visualizing and analyzing the cell culture chambers 110.

However, technical problems to be solved in the present invention are not limited to the above-described problems, and other problems which are not described herein will be fully understood by those of ordinary skill in the art from the following descriptions.

### Technical Solution

In one aspect, the present invention provides a microfluidic chip for inducing or screening anticancer drug resistant cells, which comprises: a plate 100 including a plurality of cell culture chambers 110 in a radial shape; a cell introduction part 200 formed in the central area of the plate 100 to load cells; a fluid diffusion part 300 formed along the periphery of the plate 100 to impart a space for the flow of a fluid; a first inlet 400 connected with the fluid diffusion part 300 to inject a fluid containing a culture medium and an anticancer drug; a second inlet 500 connected with the fluid diffusion part 300 to inject a fluid containing a culture medium; micro-channels 600 providing paths for the flow of fluids between the fluid diffusion part 300, the cell culture chambers 110 and the cell introduction part 200; and outlets 700 connected with the fluid diffusion part 300 to discharge the fluids outside.

In one exemplary embodiment of the present invention, the fluid diffusion part 300 is composed of a fluid diffusion part 310 connected with the first inlet and a fluid diffusion part 320 connected with the second inlet, and the first inlet-connected fluid diffusion part 310 and the second inlet-connected fluid diffusion part 320 may be spaced apart from each other.

In another exemplary embodiment of the present invention, the first inlet 400 and the second inlet 500 are located so as to face each other in the microfluidic chip.

In still another exemplary embodiment of the present invention, a continuous concentration gradient of a fluid between the cell culture chambers 110 is created in a direction of the second inlet in the microfluidic chip.

The present invention provides a method for inducing anticancer drug resistant cells using the microfluidic chip, which comprises: loading cancer cells isolated from patients into a cell introduction part 200; injecting a fluid containing a culture medium and an anticancer drug and a fluid containing a culture medium into a first inlet 400 and a second inlet 500, respectively; and forming a concentration gradient between cell culture chambers 110 by passing the fluids through micro-channels 600.

The present invention provides a method for screening anticancer drug resistant-cells using the microfluidic chip, which comprises: loading cancer cells isolated from patients into a cell introduction part 200; injecting a fluid containing a culture medium and an anticancer drug and a fluid containing a culture medium into a first inlet 400 and a second inlet 500, respectively; forming a concentration gradient between cell culture chambers 110 by passing the fluids through micro-channels 600; and real-time visualizing and analyzing the cell culture chambers 110.

In one exemplary embodiment of the present invention, the cancer cells may be human glioblastoma cells.

In another exemplary embodiment of the present invention, the anticancer drug may be doxorubicin.

### Advantageous Effects

A microfluidic chip according to the present invention can induce or screen effective anticancer drug resistant cells by creating a continuous concentration gradient between cell culture chambers. The present invention can induce rapid culture and read-out of anticancer drug resistant cells within a week unlike common techniques of inducing and reading anticancer drug resistant cells, taking at least 6 months, and is expected to be useful for the development of high-tech treatment techniques to overcome patient-customized resistance.

### DESCRIPTION OF DRAWINGS

FIG. 1 shows a basic structure of a microfluidic chip for screening anticancer drug resistant cells.

FIG. 2 is an enlarged diagram of the edge of a microfluidic chip for screening anticancer drug resistant cells, in which a fluid diffusion part, cell culture chambers, and micro-channels providing paths between the chambers are shown.

FIG. 3 shows the distribution of anticancer drug resistant cells according to a concentration of a dye (fluorescein, 1 μM) in a microfluidic chip for screening anticancer drug resistant cells, confirmed by visualization (a) and (b) a difference in fluorescence intensity.

FIG. 4 shows a visualized result of the distribution of living U87 cells in a microfluidic chip for screening anticancer drug resistant cells according to the passage of time (day 1, day 3, day 5, day 7, day 9, and day 12).

FIG. 5 shows the comparison in cell viability between U87 cells (WT cells) cultured in a general medium and U87-DG cells (chip cells) cultured using a microfluidic chip for screening anticancer drug resistant cells, according to treatment with doxorubicin.

FIG. 6 shows the comparison in redox activity between U87 cells (WT cells) cultured in a general medium and U87-DG cells (chip cells) cultured using a microfluidic chip for screening anticancer drug resistant cells.

FIG. 7 shows the comparison in Rh-123 efflux capability between U87 cells (WT cells) cultured in a general medium and U87-DG cells (chip cells) cultured using a microfluidic chip for screening anticancer drug resistant cells.

### MODES OF THE INVENTION

The present inventors cultured cancer cells using a microfluidic chip for screening anticancer drug resistant cells creating a continuous concentration gradient between cell culture chambers and compared them with those cultured in a general culture medium to confirm high cell viability due to anticancer drug resistance, and based on this, the present invention was completed.

Hereinafter, the present invention will be described in detail.

The present invention provides a microfluidic chip for inducing or screening anticancer drug resistant cells, which comprises: a plate 100 including a plurality of cell culture chambers 110 in a radial shape; a cell introduction part 200 formed in a central area of the plate 100 to load cells; a fluid diffusion part 300 formed along the periphery of the plate 100 to impart a space for the flow of a fluid; a first inlet 400 connected with the fluid diffusion part 300 to inject a fluid containing a culture medium and an anticancer drug; a second inlet 500 connected with the fluid diffusion part 300 to inject a fluid containing a culture medium; micro-channels 600 providing paths for the flow of fluids between the fluid diffusion part 300, the cell culture chambers 110 and the cell introduction part 200; and outlets 700 connected with the fluid diffusion part 300 to discharge the fluids outside.

The term “anticancer drug resistance” used herein means that in cancer treatment, an anticancer drug has no therapeutic effect from the initial treatment, or has a cancer treating effect in the initial state but loses its effect in a continuous treating process, and the inventors desired to widely use cancer cells having such anticancer drug resistance, which had been previously cultured, in basic research such as gene analysis and a clinical field such as anticancer drug resistance screening.

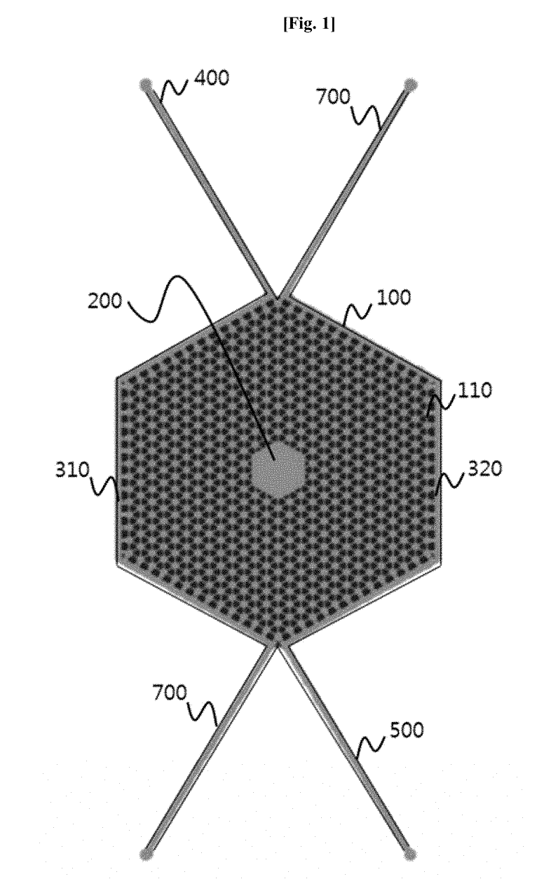

As shown in FIG. 1, the plate 100 is composed of cell culture chambers 110, a cell introduction part 200, and a fluid diffusion part 300, and the cell introduction part 200 is located in the center of the plate 100, and the fluid diffusion part 300 is formed along the periphery of the plate.

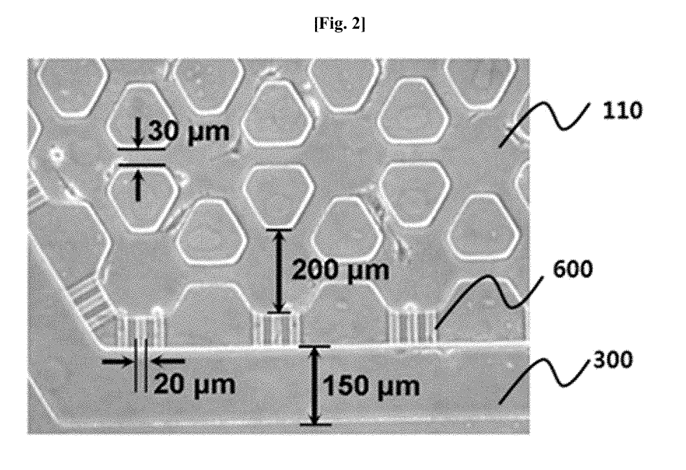

As shown in FIG. 2, the cell culture chambers 110 are spaces in which cancer cells loaded into the cell introduction part 200 formed in the middle of the plate 100, and there are 100 to 300 chambers. In addition, the cell culture chambers 110 provide a path for connecting the cell introduction part 200, the fluid diffusion part 300 and micro-channels 600 with each other via a fluid.

A fluid containing a culture medium and an anticancer drug and a fluid containing a culture medium are injected into the first inlet 400 and the second inlet 500, connected with the fluid diffusion part 300, respectively, The fluid is transferred to the fluid diffusion part 300, and then to the cell culture chambers 110 through the micro-channels 600. The first inlet 400 and the second inlet 500 are located so as to face each other, and preferably include a reservoir at each end of the inlets.

The fluid diffusion part 300 is a component for providing a space for the flow of a fluid, and as shown in FIG. 2, and a plurality of these parts may be formed along the periphery of the plate 100. Here, each of the plurality of the fluid diffusion parts 300 is preferably separated from each other. For example, the fluid diffusion parts 300 may include a fluid diffusion part 310 connected with the first inlet and a fluid diffusion part 320 connected with the second inlet, and the fluid diffusion part 310 connected with the first inlet and the fluid diffusion part 320 connected with the second inlet are spaced from each other so as not to mix the fluids injected into the first inlet 400 and the second inlet 500.

As shown in FIG. 1, the outlets 700 may be connected with the fluid diffusion part 300 to provide a path for discharging the injected fluid. In addition, as described above, there may be a plurality of the fluid diffusion parts 300, and the outlets 700 may also be plural to correspond to the fluid diffusion parts 300 spaced apart from each other. For example, the fluid diffusion part 310 connected with the first inlet and the fluid diffusion part 320 connected with the second inlet are spaced apart from each other, and here, the outlets 700 are connected with the fluid diffusion part 310 connected with the first inlet and the fluid diffusion part 320 connected with the second inlet so as to discharge an unmixed fluid through each of the outlets 700.

During such flow of fluids, a continuous concentration gradient of the anticancer drug is created. The concentration gradient is created toward the second inlet 500 into which an anticancer drug-free fluid is injected, and such a concentration gradient of the anticancer drug promotes culture and induction of anticancer drug-resistant cells.

In one exemplary embodiment of the present invention, the microfluidic chip (Death galaxy chip) of the present invention is prepared, and a continuous concentration gradient between the cell culture chambers was visualized using a dye such as fluorescein (refer to Examples 1 and 2). In addition, after U87 cells (U87-DG cells, chip cell) derived from human primary glioblastoma were cultured using the Death galaxy chip (refer to Examples 3 and 4), compared to the result with U87 cells (U87 cells, WT cell) cultured in a general medium, it is demonstrated that the U87-DG cells of the present invention exhibit excellent cell viability with respect to an anticancer drug, low redox activity, and excellent efflux capability with respect to Rh-123, and it was confirmed that the microfluidic chip of the present invention can be useful in inducing or screening anticancer drug-resistant cells (refer to Examples 5 to 7).

Therefore, the present invention provides a method for inducing anticancer drug-resistant cells using the microfluidic chip, which comprises: loading cancer cells separated from patients into a cell introduction part 200; injecting a fluid containing a culture medium and an anticancer drug, and a fluid containing a culture medium into the first inlet 400 and the second inlet 50, respectively; and creating a concentration gradient between cell culture chambers 110 after the fluids pass through micro-channels 600.

The present invention also provides a method for screening anticancer drug-resistant cells using a microfluidic chip for screening anticancer drug resistant cells, which comprises: loading cancer cells isolated from patients into a cell introduction part 200; injecting a fluid containing a culture medium and an anticancer drug and a fluid containing a culture medium into a first inlet 400 and a second inlet 500, respectively; forming a concentration gradient between cell culture chambers 110 by passing the fluids through micro-channels 600; and real-time visualizing and analyzing the cell culture chambers 110.

The term “anticancer drug” used in the inducing or screening method of the present invention is the generic term for a chemotherapeutic agent used for treatment of a tumor, preferably doxorubicin, but the present invention is not limited thereto.

In addition, anticancer drug-resistant cells targeted by induction or screening in the present invention encompass cancer cells that can acquire resistance to an anticancer drug, preferably, human glioblastoma cells, but the present invention is not limited thereto.

Hereinafter, to help in understanding the present invention, exemplary examples will be suggested. However, the following examples are merely provided to more easily understand the present invention, and the scope of the present invention is not limited to the following examples.

### Example 1. Preparation of Microfluidic Chip for Screening Anticancer Agent-Resistant Cells

A hexagonal microchamber array formed of PDMS was placed on a conventional cell culture plate. Afterward, for a strong conformal contact between surfaces, the plate was stored for several hours in a clean bench. 70% ethanol was injected into a microfluidic chip using a needle-free syringe via a cell introduction part including a central hole in the hexagonal array while being careful not to generate bubbles. 70% ethanol was sequentially changed with PBS and an MEM medium. In the following examples, a microfluidic chip for screening anticancer drug resistant cells was referred to as a Death galaxy chip.

### Example 2. Visualization of Chemical Concentration Gradient in Death Galaxy Chip

After a Death galaxy chip was prepared, a central cell introduction part was covered with a cover glass (10 mm Φ) (The Paul Marienfeld GmbH & Co KG., Lauda-Konigshofen, Germany). A pair of inlets located to face each other were charged with a dye (fluorescein, 1 μM)-containing MEM medium (300 μl) and a dye-free MEM medium (300 μl), respectively. Six hours later, a fluorescent image was obtained using a stereomicroscope (Olympus Corporation, Tokyo, Japan), and a fluorescence intensity of the entire culture chip was analyzed with ImageJ software.

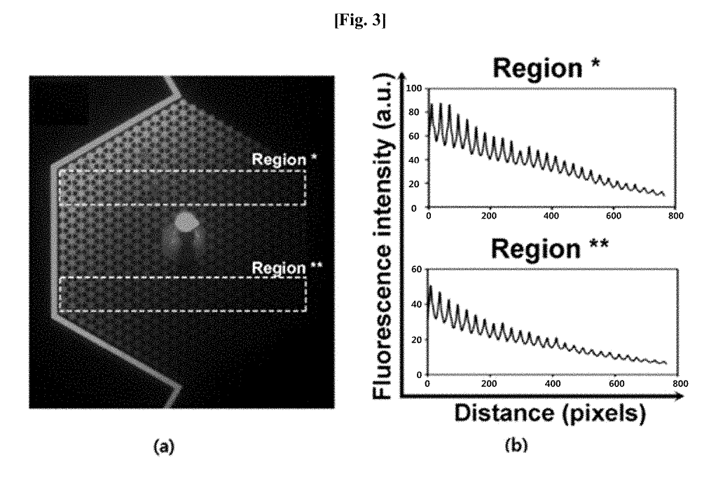

As a result, as shown in FIG. 3, a cell culture chamber near the inlet into which the dye-containing MEM medium (300 μl) was injected exhibited high fluorescence intensity, and the inlet opposite to the previous one exhibited low fluorescence intensity, and it was confirmed that a concentration gradient was created in a direction of the dye-free MEM medium (300 μl)-injected inlet with the flow of the dye through micro-channels.

### Example 3. Cell Culture in Death Galaxy Chip

Approximately 5000 cells in MEM media were loaded into a Death galaxy chip using a pipette via the central cell introduction part of the hexagonal array, and then covered with a cover glass.

A pair of inlets located so as to face each other were charged with MEM media, and then the cells were cultured in a cell culture incubator. After twenty-four hours of culturing, the media were removed from the inlets, and one of the inlets was charged with only an MEM medium (300 μl), and the other thereof was charged with a doxorubicin (5 μM)-containing MEM medium (300 μl). Afterward, the cells were cultured for 7 days or more, and every 12 hours, fresh media and drugs were injected into the inlets, and then wastes accumulated in the two inlets were removed.

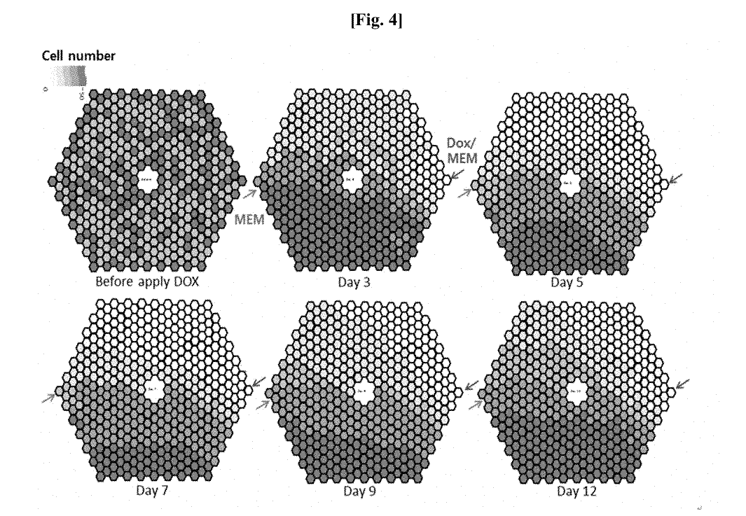

As a result, as shown in FIG. 4, it was confirmed that the number of cells were reduced near the doxorubicin (5 μM)-containing MEM medium-injected inlet according to a concentration gradient of doxorubicin, and the number of living cells were increased close to the MEM medium (300 μl)-containing inlet. Accordingly, the result shows that anticancer drug-resistant cells can be screened and cultured using the Death galaxy chip of the present invention.

### Example 4. Yield of Anticancer Agent-Resistant Cells (U87-DG Cells)

In the Death galaxy chip, for 7 days, U87 cells derived from human primary glioblastoma were exposed to an anticancer drug such as doxorubicin according to a concentration gradient, and a hexagonal microchamber array was removed from a surface of the living cells (U87-DG cells)-attached cell culture plate. The inventors trypsinized the U87-DG cells to analyze the characteristic of phenotypes thereof, collected the cells, and transferred them to a new culture plate to culture the cells in a normal growth medium (MEM) for one week.

### Example 5. Evaluation of Anticancer Agent Resistance

3,000 cells (U87 and U87-DG cells) were seeded into a 96-well plate in 200 μl of an MEM medium containing doxorubicin at various concentrations (0, 0.005, 0.01, 0.02, 0.05, 0.1, 0.2, 0.5, 0.75, and 1 μM). After seventy-two hours of culturing, the number of living cells in each well was calculated. Cell viability was calculated by dividing the number of living cells in a doxorubicin-contained well by the number of living cells in a doxorubicin-free well. After plotting a concentration of doxorubicin with respect to cell viability, IC₅₀ was estimated through non-linear regression analysis using Graphpad Prism (GraphPad Software, Inc., La Jolla, Calif. USA).

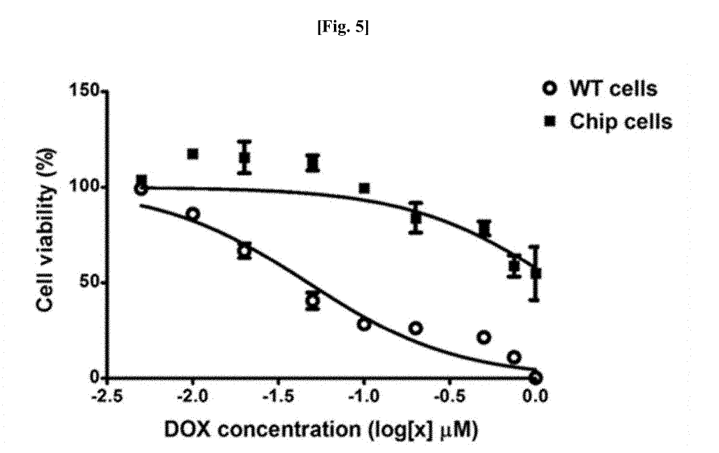

As a result, as shown in FIG. 5, it was confirmed that, compared to U87 cells (WT cells), cell viability of U87-DG cells (chip cells) cultured with the Death galaxy chip was significantly increased, the IC₅₀ of the U87 cells was 0.05 μM, and the IC₅₀ of the U87-DG cells was 1.47 μM. The result shows that U87 cells having doxorubicin resistance can be effectively obtained by providing a culture condition in which a concentration gradient of doxorubicin is created.

### Example 6. Confirming Mitochondrial Redox Activity

Mitochondrial redox capacities of U87 cells and U87-DG cells were compared using an EZ-Cytox assay kit (Daeil Lab Service, Seoul, Korea). The cells were seeded into a 96-well plate in 180 μl of a culture medium, and cultured for 6 hours. A WST reagent (20 μl) was added to each well, and cultured for 1 hour. The absorbance of WST-formazan produced by a mitochondrial dehydrogenase was measured using a microplate reader (Molecular Devices Inc. Sunnyvale, Calif.) at 450 nm.

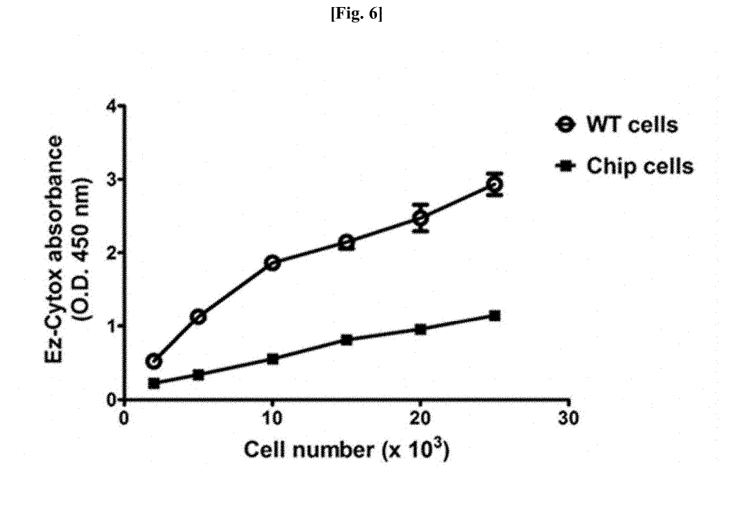

As a result, as shown in FIG. 6, compared to U87 cells (WT cells), U87-DG cells (chip cells) showed a low absorbance. The result shows that mitochondrial aerobic respiration in the U87 cells can be reduced by culturing the cells with the Death galaxy chip.

### Example 7. MDR Efflux Assay

Multi-drug resistance was measured by evaluating an efflux capability of a fluorescent dye Rh-123 (rhodamine 123) (Sigma-Aldrich, St. Louis, Mo., USA) to substrates for MDR1 and MRP1 proteins. Cells were preloaded with Rh-123 on ice for 30 minutes, and then incubated in a 37° C. water bath to induce MDR protein-mediated efflux of the dye. The cells were stained with propidium iodide (PI; Sigma-Aldrich, St. Louis, Mo., USA), and stored on ice until being used in analysis. The discharged dye was analyzed through flow cytometry (BD Biosciences, San Jose, Calif., USA), and PI-positive dead cells were excluded from the analysis. Meanwhile, the release of Rh-123 was analyzed using a fluorometric plate reader (Molecular Devices Inc. Sunnyvale, Calif.). A cell stop solution was divided into each well of a 96-well plate before and after the dye efflux. Fluorescence intensities were measured at an excitation wavelength of 485 nm and an emission wavelength of 530 nm.

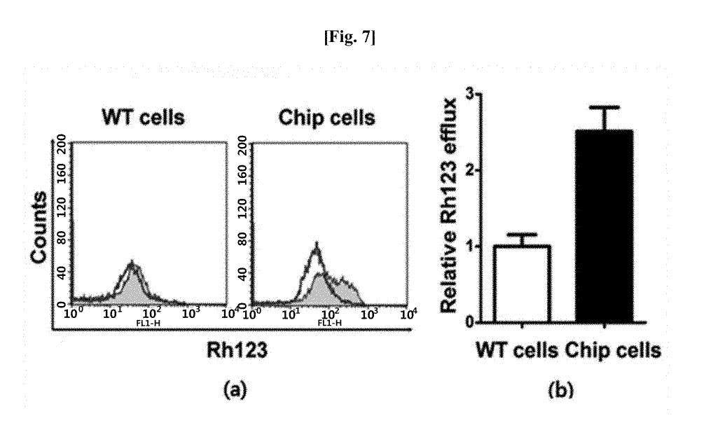

As a result, as shown in FIG. 7, it was confirmed that, compared to U87 cells (WT cells), U87-DG cells (chip cells) have an excellent efflux capability of MDR1 and MRP1 proteins. The result shows that, by culturing cells with the Death galaxy chip, efflux pumps were further activated, resulting in reduction of a drug accumulation rate in the U87 cells and attainment of resistance to doxorubicin.

It would be understood by those of ordinary skill in the art that the above description of the present invention is exemplary, and the exemplary embodiments disclosed herein can be easily modified into other specific forms without departing from the technical spirit or essential features of the present invention. Therefore, the exemplary embodiments described above should be interpreted as illustrative and not limited in any aspect.

### INDUSTRIAL APPLICABILITY

A microfluidic chip according to the present invention is able to induce or screen effective anticancer drug-resistant cells by creating a continuous concentration gradient between cell culture chambers. The present invention is able to induce rapid culture and read-out of anticancer drug resistant cells within a week unlike common techniques of inducing and reading anticancer drug resistant cells, taking at least 6 months, and is expected to be useful for the development of high-tech treatment techniques to overcome patient-customized resistance.

1. A microfluidic chip for screening or inducing anticancer drug resistant cells, comprising:
a plate including a plurality of cell culture chambers in a radial shape;
a cell introduction part formed in a central area of the plate to load cells;
a fluid diffusion part formed along a periphery of the plate to impart a space for the flow of a fluid;
a first inlet connected with the fluid diffusion part to inject a fluid containing a culture medium and an anticancer drug;
a second inlet connected with the fluid diffusion part to inject a fluid containing a culture medium; micro-channels which provide paths for the flow of fluids between the fluid diffusion part, the cell culture chambers and the cell introduction part; and
outlets connected with the fluid diffusion part to discharge the fluids outside.

2. The microfluidic chip of claim 1, wherein the fluid diffusion part is composed of a fluid diffusion part connected with the first inlet and a fluid diffusion part connected with the second inlet, and the first inlet-connected fluid diffusion part and the second inlet-connected fluid diffusion part are separated from each other.

3. The microfluidic chip of claim 1, wherein the first inlet and the second inlet are located so as to face each other.

4. The microfluidic chip of claim 1, wherein a continuous concentration gradient of a fluid between the cell culture chambers is created in a direction of the second inlet.

5. A method for inducing anticancer drug resistant cells using the microfluidic chip according to claim 1, the method comprising:
loading cancer cells isolated from patients into a cell introduction part; injecting a fluid containing a culture medium and an anticancer drug and a fluid containing a culture medium into a first inlet and a second inlet, respectively; and forming a concentration gradient between cell culture chambers by passing the fluids through micro-channels.

6. The method of claim 5, wherein the cancer cells are human glioblastoma cells.

7. The method of claim 5, wherein the anticancer drug is doxorubicin.

8. A method for inducing anticancer drug resistant cells using the microfluidic chip according to claim 1, the method comprising:
loading cancer cells isolated from patients into a cell introduction part; injecting a fluid containing a culture medium and an anticancer drug and a fluid containing a culture medium into a first inlet and a second inlet, respectively; forming a concentration gradient between cell culture chambers by passing the fluids through micro-channels; and real-time visualizing and analyzing the cell culture chambers.

9. The method of claim 8, wherein the cancer cells are human glioblastoma cells.

10. The method of claim 8, wherein the anticancer drug is doxorubicin.
